(19) United States
(12) Patent Application Publication
(10) Pub. No.: US 2023/0414557 A1
(43) Pub. Date: Dec. 28, 2023

(54) NEW THERAPY CONCEPT FOR THE TREATMENT OF CORONA INFECTIONS, ESPECIALLY COVID-19 INFECTIONS

(71) Applicant: Maria Clementine Martin Klosterfrau Vertriebsgesellschaft mbh, Koln (DE)

(72) Inventor: Michael PLOCH, Koln (DE)

(21) Appl. No.: 18/252,962

(22) PCT Filed: Oct. 6, 2021
(86) PCT No.: PCT/EP2021/077544

§ 371 Date: May 15, 2023

(30) Foreign Application Priority Data
Nov. 18, 2020 (DE) 10 2020 007 038.8
Nov. 30, 2020 (DE) 10 2020 131 716.6

(51) Int. Cl. A61K 31/352; A61K 47/14; A61K 45/06; A61P 31/14

(52) U.S. Cl. CPC A61K 31/352; A61P 31/14; A61K 45/06; A61K 47/14

(57) ABSTRACT

The present invention relates to the medical-pharmaceutical field of therapy of viral infections or viral diseases caused by corona viruses, especially COVID-19; especially, the present invention relates to an active ingredient and a drug or medicament containing said active ingredient for use in such therapy.

### CROSS-REFERENCES TO RELATED APPLICATIONS

This application is a National Stage filing of International Application Application PCT/EP 2021/077544 (WO 2022/106112) filed Oct. 6, 2021, entitled “NEW THERAPY CONCEPT FOR THE TREATMENT OF CORONA INFECTIONS, MORE PARTICULARLY COVID-19 INFECTIONS” claiming priority to DE 10 2020 007 038.8 filed Nov. 18, 2020, and DE 10 2020 131 716.6 filed Nov. 30, 2020. The subject application claims priority to DE 10 2020 007 038.8, DE 10 2020 131 716.6, and PCT/EP 2021/077544, and incorporates all by reference herein, in their entirety.

### BACKGROUND OF THE INVENTION

The present invention relates to the technical (i.e. medical-pharmaceutical) field of therapy of viral infections, especially infections with corona viruses, especially COVID-19. Especially, the present invention relates to an active ingredient and a pharmaceutical composition for use in such therapy.

Especially, the present invention relates to an active ingredient and a drug or composition, respectively, for use in the prophylactic and/or therapeutic treatment of viral infections caused by corona viruses (hereinafter also referred to as “corona infections”, “corona(virus) infections” or the like), especially COVID-19, or to the use of an active ingredient and a medicament or a composition for the prophylactic and/or therapeutic treatment of viral infections caused by corona viruses, especially COVID-19. In the context of the present invention, cineole, preferentially 1,8-cineole, or a medicament or composition containing this active ingredient, respectively, is used as the active ingredient, as further defined and described in the following description and in the patent claims relating to the present invention.

Coronaviridae is a family of viruses within the order Nidovirales. The viruses within this virus family are also known colloquially as coronaviruses and are among the RNA viruses with the largest genomes. The first coronaviruses were discovered and described as early as the mid-1960s. The roughly spherical viruses in the electron microscope image are conspicuous by a ring of petal-like projections reminiscent of a solar corona, which gave this virus family its name.

Representatives of the Coronaviridae family of viruses cause very different diseases in all four classes of terrestrial vertebrates (i.e. mammals, birds, reptiles, and amphibians). They are genetically highly variable and thus can also infect multiple species of hosts.

In humans, seven types of coronaviruses are important as pathogens of mild respiratory infections (especially colds or flu-like infections) up to the so-called severe acute respiratory syndrome (SARS or Severe Acute Respiratory Syndrome).

Among human coronaviruses, the following coronaviruses have become particularly well known: SARS-CoV-1 (Severe Acute Respiratory Syndrome Coronavirus-1), MERS-CoV (Middle East Respiratory Syndrome Coronavirus), and SARS-CoV-2 (Severe Acute Respiratory Syndrome Coronavirus 2 or COVID-19). These coronaviruses are the triggers of the 2002/2003 SARS pandemic, the 2012 MERS epidemic, and the 2019 COVID-19 pandemic, respectively.

COVID-19 (i.e. the abbreviation for English Coronavirus Disease 2019 or German Coronavirus-Krankheit-2019, colloquially also referred to as coronavirus disease/infection or the like) is an infectious disease resulting in infection with the novel coronavirus SARS-CoV-2. Primarily affecting the respiratory tract, the disease was first described in Wuhan, China, in late 2019, then developed into an epidemic first in the People's Republic of China in January 2020, and eventually spread globally to become the COVID-19 pandemic.

Infection with COVID-19 usually occurs through droplet transmission. The incubation period of COVID-19 is on average five to six days, although up to two weeks can pass between infection and the appearance of the first symptoms, and in isolated cases the first symptoms can appear within 24 hours of infection with SARS-CoV-2. The most common symptoms are fever, dry cough, and fatigue; less common symptoms include muscle pain, nasal congestion, headache, conjunctivitis, sore throat, diarrhea, loss of taste or smell, or skin rash or discoloration of fingers or toes. Infected individuals without symptoms may still be potential carriers of coronavirus. If the course of the disease is mild, symptoms generally resolve within two weeks. If COVID-19 is more severe, convalescence may last three to six weeks or even longer.

In about 81% of registered infections, the course of the disease is mild, with fever or mild pneumonia; however, in about 14% of cases, the course is more severe and in about 5% of cases, the course is even so severe that patients require intensive care.

COVID-19 is currently the subject matter of intensive research. Efficient and specific causal therapies or vaccinations are currently not available or only to a limited extent.

Like SARS-CoV-1 in SARS, the COVID-19-causing virus SARS-CoV-2 typically enters the human cell via binding to the cell membrane-anchored enzyme ACE2 (angiotensin-converting enzyme 2), whereby the viral spike protein interacts with ACE2. This method requires the involvement of the serine protease TMPRSS2 (transmembrane serine protease 2). In experiments with HeLa cells expressing ACE2 from humans, Chinese horseshoe bats (Rhinolophus sinicus), a creeping cat species, domestic pigs and mice, SARS-CoV-2 was able to use the respective ACE2 protein as a receptor to enter the cell; only the mouse ACE2 enzyme failed to do so—as did HeLa cells, which do not produce ACE2. In contrast, SARS-CoV-2 does not bind to receptors used by other coronaviruses.

In addition to the pathway via ACE2, an alternative penetration pathway has been experimentally demonstrated in T-lymphocytes that carry little or no ACE2 on their surface: The virus penetrates these cells via a spike protein-mediated fusion of the viral membrane with the lymphocyte cell membrane.

A reverse search in a human gene database (Human Cell Atlas or HCA for short) for cell types and tissues in which, in addition to ACE2, TMPRSS₂ is also present on membrane surfaces shows that the highest concentrations of these two proteins occur in the nasal mucosa, especially in the goblet cells, but also in the ciliated epithelia, which is why these cells are regarded as a portal of entry for SARS-CoV-2 and are also suspected of being a reservoir. The proteins are also produced in the corneal cells of the eye, in the intestinal mucosa and in the heart in pericytes of the blood capillaries, cardiac muscle cells and fibroblasts. The first phase of the infection in the nasopharynx remains almost symptom-free, whereas the lungs are predominantly attacked when the disease progresses to a severe form, since a large proportion of the ACE-2-expressing cells in humans are found in the type II pneumocytes of the lungs. Other reasons given for the particular susceptibility of the lung are its large surface area; in addition, ACE-2-expressing pneumocyte type II cells possess diverse genes that favor replication and transmission of SARS-CoV-2.

In studies of cryopreserved lung tissue samples from non-infected individuals, it can also be shown that lung tissue hardly produces ACE2 as well as the transmembrane protease TMPRSS2, whereas pneumocytes type II in the lung are increased. These progenitor cells tend to be increased in males and in advanced age. In addition to different ACE2 levels in men and women, one reason for the different severity of the disease is suspected to be the gender-specific hormone balance: estrogen promotes an immune response, whereas testosterone suppresses it.

Recent evidence also shows that the proprotease furin is co-expressed in the lung epithelium and adjacent tissue cells, which in turn facilitates cell entry for the virus by providing a furin-specific separation site at the spike protein.

In addition to the lungs, ACE2 has also been detected in the small and large intestines, the respiratory tract, and the kidneys. Multiplication of the virus in intestinal cells was also confirmed.

In terms of clinical symptoms and laboratory signs of illness, it is difficult to distinguish it from other viral diseases, such as influenza, on the basis of symptoms alone. After an incubation period of typically five to six days, in rare cases up to 14 days, fever, muscle pain and a dry cough may occur—as described at the beginning. Frequently, the disease also manifests itself with a general feeling of severe illness. In the further course, severe shortness of breath may then develop due to infection of the lower respiratory tract up to pneumonia. This may be accompanied by chest pain in the form of pleurisy. Approximately 85% of severely ill COVID-19 patients develop lymphopenia. The severely ill patients also frequently develop hypercytokinemia (so-called cytokine storm), whereby this cytokine storm results from an overreaction of the immune system; this overreaction is characterized by a marked increase in inflammation-relevant cytokines, such as interleukin-6, interleukin-8, interleukin-1beta and TNF-alpha especially. The increased release of these cytokines leads to an overproduction of immune cells, especially in the lung tissue.

Especially, the diagnosis of COVID-19 can be made by laboratory diagnostic detection, especially using specific viral and antibody detections.

An efficient and specific causal therapy has not yet been established. The nucleoside analog Remdesivir shows a reduction of disease duration in hospitalized patients in a preliminary published randomized study. This drug is approved in the European Union for COVID-19 patients requiring oxygen and can be considered in critically ill patients according to the DIVI (Deutsche Interdisziplinäre Vereinigung für Intensiv- und Notfallmedizin e.V.) guideline.

Further studies show that dexamethasone (9-fluoro-16alpha-methylprednisolone) reduces mortality from 41% to 29% in patients on ventilators and from 26% to 23% in patients on oxygen. Dexamethasone is able to prevent or reduce an excessive reaction of the immune system, especially the so-called cytokine storm.

In severely ill COVID-19 patients, administration of low molecular weight heparin is also recommended to reduce the risk of thrombosis and pulmonary embolism. Due to the high incidence of pulmonary embolism and leg vein thrombosis associated with severe courses of COVID-19, the administration of more potent anticoagulants is being considered; however, sufficient data on benefit and risk are not yet available.

Chloroquine and hydroxychloroquine, on the other hand, show no evidence of efficacy-contrary to original expectations.

Tocilizumab, a monoclonal antibody approved for the treatment of various forms of rheumatoid arthritis and cytokine release syndrome, among others, has also been shown to be ineffective.

Antibody-rich plasma from recovered patients, on the other hand, appears to be suitable for treating acute cases, but can demonstrate success only in the early phase of the disease.

To date, it is also still unclear to what extent a passed-through infection confers immunity; so far, based on findings from animal models, it is assumed that acute immunity exists, although it is not certain how long it lasts. The state of research on late effects and estimation of mortality risk is also still uncertain.

As a result, most measures worldwide are therefore aimed at prevention of COVID-19 diseases, especially through increased individual hygiene measures, such as wearing oral-nasal protection, hand disinfection, surface cleaning etc., since no established causal therapy for curative treatment or vaccination for prevention purposes exists yet.

Against this background, one object of the present invention is to provide an efficient therapy for viral diseases or viral infections (viral infections) triggered by corona viruses, especially COVID-19.

Especially, it is an object of the present invention to find or provide a suitable active ingredient and a drug or composition containing said active ingredient, which is suitable for use in the prophylactic and/or therapeutic treatment of corona virus-induced viral diseases or corona virus-induced viral infections (i.e. corona (virus) infections), especially COVID-19, preferably in the context of an efficient therapy.

### BRIEF SUMMARY OF THE INVENTION

In a completely surprising manner, the applicant has now discovered that cineole, especially 1,8-cineole, is unexpectedly and efficiently suitable as an active ingredient for the therapy of viral diseases caused by corona viruses or viral infections caused by corona viruses (i.e. corona (virus) infections), especially COVID-19.

To solve the problem described above, the present invention therefore proposes—according to a first aspect of the present invention—cineole, especially 1,8-cineole, for use in the prophylactic and/or therapeutic treatment of viral diseases caused by corona viruses (i.e., corona virus infections), especially COVID-19. Advantageous further developments and embodiments of this aspect of the invention are discussed below.

A further subject matter of the present invention—according to a second aspect of the present invention—is the use according to the invention of cineole, preferentially 1,8-cineole, for the prophylactic and/or therapeutic treatment of viral diseases caused by corona viruses, especially COVID-19, or the use of cineole, preferentially 1,8-cineole, according to the invention for producing of a pharmaceutical or medicament for the prophylactic and/or therapeutic treatment of viral diseases caused by corona viruses, especially COVID-19, respectively. Advantageous further developments and embodiments of this aspect of the invention are provided.

Equally, the subject matter of the present invention—according to a third aspect of the present invention—is the use of cineole, preferentially 1,8-cineole, according to the invention as an antiviral agent for the prophylactic and/or therapeutic treatment of viral diseases caused by corona viruses, especially COVID-19, or for producing an antiviral agent for the prophylactic and/or therapeutic treatment of viral diseases caused by corona viruses, especially COVID-19, respectively. Advantageous further developments and embodiments of this aspect of the invention are described below.

Yet another subject matter of the present invention—according to a fourth aspect of the present invention—is a method for the prophylactic and/or therapeutic treatment of viral diseases caused by corona viruses, especially COVID-19.

Also a subject matter of the present invention—according to a fifth aspect of the present invention—is a medicament or drug for (use in the) prophylactic and/or therapeutic treatment of viral diseases caused by corona viruses, especially COVID-19, especially an antiviral agent.

Similarly, it is a subject matter of the present invention—according to a sixth aspect of the present invention—to provide a composition for (use in the) prophylactic and/or therapeutic treatment of viral diseases caused by corona viruses, especially COVID-19.

Finally, the subject matter of the present invention—according to a seventh aspect of the present invention—is equally a pharmaceutical combination for (use in the) prophylactic and/or therapeutic treatment of viral diseases caused by corona viruses, especially COVID-19.

It goes without saying that aspects, embodiments, advantages and the like, which are listed below for the purpose of avoiding repetition with respect to one aspect of the invention, naturally also apply accordingly with respect to the other aspects of the invention, without this requiring separate mention.

Furthermore, with regard to the following description of the present invention, it is also the case that the features of the present invention cited in each case in connection with the specific embodiments, embodiments, advantages, examples or the like are also deemed to be disclosed in their combination. Thus, superordinate combinations of individual or several features, which are indicated for respective embodiments, embodiments, examples of use or the like, are also considered disclosed herein.

Furthermore, it goes without saying that in the following statements of values, numbers and ranges, the relevant statements of values, numbers and ranges are not to be understood as restrictive; it goes without saying for the person skilled in the art that, depending on the individual case or application, deviations from the stated range or statements can be made without leaving the scope of the present invention.

In addition, all values or parameters or the like mentioned in the following can basically be determined using standardized or explicitly specified determination methods or using determination methods that are familiar to the skilled person in this field.

Furthermore, it should be noted with regard to all the relative or percentage, especially weight-related, quantitative data mentioned below that, within the framework of the composition according to the invention, these data are to be selected or combined by the person skilled in the art in such a way that in the sum—given the inclusion of further components or ingredients or additives substances or constituents, especially as defined below—always results in 100% or 100 wt. %. However, this is self-evident for the person skilled in the art.

### DETAILED DESCRIPTION OF THE INVENTION

Thus, the subject matter of the present invention—according to a first aspect of the present invention—is a cineole, preferentially 1,8-cineole, for use in the prophylactic and/or therapeutic treatment of viral diseases caused by corona viruses, especially COVID-19 (i.e. in other words, the use of a cineole, preferentially 1,8-cineole, for the prophylactic and/or therapeutic treatment of viral diseases caused by corona viruses, especially COVID-19).

This is because—as previously stated—the applicant has found, in a completely surprising way, that cineole, especially 1,8-cineole, is unexpectedly and efficiently suitable as an active ingredient for the therapy of viral diseases caused by corona viruses or of viral infections caused by corona viruses (i.e. corona (virus) infections), especially COVID-19.

This special and new as well as inventive application or medical indication for cineole, especially 1,8-cineole, surprisingly discovered by the applicant in the context of the present invention, has not yet been described in the prior art and has not even been recognized, although cineole, especially 1,8-cineole, is in itself a sufficiently known active substance.

The active ingredient cineole used according to the invention, especially 1,8-cineole, is a so-called terpene, especially a monoterpene. Terpenes are generally natural substances which can be isolated from plants or their constituents as components of so-called essential oils in the form of liquids. They are often fragrances and flavorings, which are used in the food or cosmetics industry. However, the use of terpenes for medical purposes is also gaining importance, since pharmacological effects can be demonstrated for a large number of terpenes. Terpenes are formally polymerization products of isoprene, whereby a distinction is made between monoterpenes (C₁₀-units), sesquiterpenes (C₁₅-units), diterpenoids (C₂₀-units), sester terpenes (C₂₅-units), triterpenes (C₃₀-units), tetraterpenes (C₄₀-units) and polyterpenes according to the number of isoprene residues (cf. ROMPP-Chemielexikon, 10th edition, Georg Thieme Verlag, Stuttgart/New York, 1999, pages 4449 and 4450, keyword “Terpen(oid)e”). In addition, terpenes can also be used as pharmacologically active substances, starting materials for the production of drugs or vitamin preparations, and in agriculture due to their often bacteriocidal or pesticidal effects. Especially for a number of monoterpenes pharmacological effects in the treatment of diseases in the case of systemic treatments have been proven, especially menthol and cineole, especially 1,8-cineole.

Especially, the active ingredient cineole used according to the invention, especially 1,8-cineole, belongs to the bicyclic epoxy monoterpenes, more specifically the limonene oxides. Synonymous designations for 1,8-cineole with the chemical molecular formula C₁₀H₁₈O are eucalyptol, limonene-1,8-oxide, 1,8-epoxy-p-menthane or 1,3,3-trimethyl-2-oxabicyclo[2.2.2]octane. It is a colorless liquid with a spicy, camphor-like odor with a melting point of +1.5° C. and a boiling point of 176 to 177° C., which is insoluble in water but miscible with most organic solvents.

Of course, 1,8-cineole occurs as the main component of eucalyptus oil (eucalyptus oil contains up to 85% by weight of 1,8-cineole), but it is also found in other plants, such as mint, medicinal sage, thyme, basil and tea tree. In addition, 1,8-cineole is also present, for example, in niaouli, juniperus, piper, cannabis, cajeput, sage oil, myrtle oil and other essential oils.

Technically or pharmaceutically purified 1,8-cineole, which can generally be present at 99.6% to 99.8% purity, is generally obtained by fractional distillation of eucalyptus oil.

In the prior art, 1,8-cineole is used especially as an expectorant for bronchial catarrh and other respiratory diseases, but also as a flavoring agent in the perfume industry. Furthermore, 1,8-cineole is used in dentistry for the revision of root fillings. In physiological terms, 1,8-cineole has an expectorant effect in the upper and lower respiratory tract, especially in the lungs and sinuses. According to the state of the art, 1,8-cineole is applied both topically (e.g. inhalatively) and systemically (e.g. in the form of capsules), usually as a mixed oil together with a variety of other terpenes.

For further details on the active ingredient 1,8-cineole, reference can be made, for example, to ROMPP Chemielexikon, Georg Thieme Verlag, Stuttgart/New York, 10th edition, Volume 1, 1996, page 752, keyword: “Cineole”, and the literature referenced therein.

Completely surprising and unexpected, however, is the antiviral action potential of cineole, especially 1,8-cineole, found by the applicant in the context of the present invention in the prophylactic and/or therapeutic treatment of viral diseases caused by corona viruses, especially COVID-19.

As the applicant has surprisingly found in this context, platelets, which in addition to blood clotting also function as immunoregulatory inflammatory cells, can harbor coronaviruses and thus trigger viral infections triggered by coronaviruses, especially COVID-19, and surprisingly, cineole is able to counteract a hyperinflammatory syndrome occurring in the course of coronavirus infection and, above all, hypercytokinemia (cytokine storm) and to contain the coronavirus infection.

In previous COVID-19 infection courses, it is known that especially and in principle two states of inflammation can occur, namely the one leading to hyperinflammation with a potentially critical course or the one leading to viral clearance.

Primarily, platelets are known from blood coagulation and maintenance of vascular integrity; however, recent studies also show that platelets are important active regulators of the immune system and are particularly attributable to the innate immune system. In this context, the importance of platelets in inflammation-related diseases has been demonstrated, thus providing insights into pathomechanisms.

In the context of viral infections, platelets are particularly capable of inducing and maintaining massive inflammatory responses (especially hypercytokinemia) as well as presenting antigens and initiating immune responses in viral diseases, but also of taking up RNA and passing it horizontally to other cells and, moreover, of taking up RNA viruses and providing them with a replication site. Furthermore, platelets are able to produce so-called platelet-derived microvesicles or PMPs and regulate them through PMP immune cells.

Based on clinical observations in COVID-19 infections, thrombocytes can be considered or evaluated as central target cells of COVID-19 infection: Thus, an altered platelet/lymphocyte ratio (so-called platelet-to-lymphocyte ratio or PTR) can be used as an inflammatory marker and prognostic factor with respect to the course of COVID-19 infections. Altered coagulation behavior also suggests that PTL inhibition is associated with a milder course in COVID-19 infections. Thrombocytopenia, on the other hand, may be considered a marker for a severe COVID-19 course. COVID-19 infections are generally characterized by a variety of proinflammatory effector cytokines released from platelets, such as TNF, IL-1(3, IL-6, IL-8, G-CSF und GM-CSF.

Surprisingly, in the context of the present invention, the applicant was able to gain a fundamental clinical and immunological understanding regarding the alteration of thrombocytes in the context of COVID-19 infections under cineole treatment.

In this context, it has been shown by the applicant that cineole, especially 1,8-cineole, can be used efficiently in the treatment of viral diseases caused by corona viruses, especially COVID-19.

For example, in vitro studies with thrombocytes obtained from healthy volunteers, which were first incubated with 1,8-cineole and then infected with corona viruses, showed that an outbreak of infection, especially hypercytokinemia, can be prevented or reduced. In further in vitro studies with thrombocytes obtained from patients suffering from COVID-19, which were incubated with 1,8-cineole, these results could be confirmed, especially to the effect that the course of infection, especially hypercytokinemia, can be prevented or attenuated. This antiviral effect of 1,8-cineole in COVID-19 infections is completely unexpected.

In this context, it is known that platelets adhere to lung epithelia and in this way enable or exacerbate infection of the epithelia with COVID-19. Adhesion to epithelia is in turn cytokine-dependent and consequently can be inhibited or reduced or attenuated by cineole, especially 1,8-cineole. This also mitigates or prevents infection of the epithelia and ultimately the lung tissue.

This effect, which the applicant found completely surprising, can possibly—without wanting to commit to a specific theory—also be attributed to the fact that a systemic, especially peroral, administration of cineole, especially 1,8-cineole, leads to the fact that, especially as a result of diffusion and/or exhalation methods, a certain part of the systemically or perorally applied cineole reaches the respiratory epithelium, especially the alveolar and/or bronchial epithelium, unchanged and is exhaled there, so to speak, and is consequently found in relatively high concentrations on and in the epithelial cells, so that it can develop its effect there in an efficient manner. This is of particular advantage in the context of a therapy according to the invention of viral infections caused by corona viruses (viral infections), especially COVID-19, with cineole, especially 1,8-cineole, especially since an inflammation of the lung epithelium occurs in a large number of the infections.

As a result of the lipophilicity of the systemically or perorally applied cineole, especially 1,8-cineole, long-term storage in the epithelial cells concerned is also observed, so that a long-term and uniform supply of the cineole, especially 1,8-cineole, in the epithelial cells concerned is also ensured. This is also particularly advantageous with regard to a treatment according to the invention of viral infections caused by corona viruses, especially COVID-19.

The efficacy of the cineole, especially 1,8-cineole, used according to the invention in the treatment of viral infections caused by corona viruses (viral infections), especially COVID-19, can possibly also be explained—again without wishing to commit to a specific theory—by the steroid-like action potential of the cineole, especially 1,8-cineole, used, which was surprisingly discovered by the applicant, especially with regard to the inhibition of inflammatory mediators, i. e. cineole, especially 1,8-cineole, especially as a pure substance, thus possesses a steroid-like action potential and is capable of causing the inhibition of inflammatory mediators. Essential mixed oils, on the other hand (which contain cineole in mixture with further terpenes and other active substances), stimulate prostaglandin production and show only a reduced inhibition of leukotriene and cytokine production compared to pure cineole, especially pure 1,8-cineole; because such mixed oils also contain such substances which stimulate cell activity and mediator production and therefore do not have an anti-inflammatory effect, but can cause intolerance reactions, so that essential mixed oils and/or oil mixtures consequently generally even increase cell activity and can induce inflammatory mediator production. In contrast to this, however, cineole, especially 1,8-cineole, especially in pure form, causes a significant inhibition of mediator production; this thus causes an anti-inflammatory effect. This effect is achieved with systemic application of cineole, especially 1,8-cineole, in the entire body and especially in the entire respiratory tract, especially in the entire lung, i.e. also as far as the periphery and the alveoli. This is because cineole, especially 1,8-cineole, is absorbed into the blood after systemic administration (especially in the form of capsules that are entericcoated (=resistant to gastric juice) but soluble in the small intestine) and is released into the alveoli, among other places, according to its physical properties. Through this, the cineole, especially 1,8-cineole, can impart an anti-inflammatory effect throughout the body and especially in the smallest, peripherally located airways.

Due to this profile of action, cineole, especially 1,8-cineole, is suitable in the prophylactic and/or therapeutic treatment of viral diseases caused by corona viruses, especially COVID-19, particularly as a co-therapeutic to corticosteroids, especially dexamethasone: As the applicant surprisingly found, cineole, especially 1,8-cineole, is also able to improve the effectiveness or efficiency of corticosteroids (e.g. especially dexamethasone) administered systemically in the treatment of viral diseases caused by corona viruses, especially COVID-19, significantly, especially in a synergistic manner and to significantly reduce the doses used, combined with the associated advantages (e.g. avoidance or reduction of side effects etc.). At the same time, the interaction of cineole, especially 1,8-cineole, on the one hand and of especially systemically administered corticosteroids (e.g. especially dexamethasone) on the other hand significantly increases the sensitivity to the especially systemically administered corticosteroids (e.g. especially dexamethasone); consequently, the dosage of the corticosteroids (e.g. especially dexamethasone) which are especially systemically administered can be reduced if necessary.

Through investigations on the part of the applicant, it was surprisingly found that cineole, especially 1,8-cineole, is capable of increasing the effect of corticosteroids (e.g. especially dexamethasone) in a significant, especially synergistic manner. Surprisingly, it was found that therapeutically relevant concentrations of cineole, especially 1,8-cineole, together with corticosteroids (e.g. especially dexamethasone) can cause a synergistic inhibition of cytokine production and consequently significantly enhance the anti-inflammatory effect of corticosteroids.

As the applicant was able to show in in vitro studies on human monocyte cultures, 1,8-cineole alone significantly inhibits the LPS-stimulated production of IL-1beta. In combination with a corticosteroid (e.g. especially dexamethasone), 1,8-cineole can synergistically enhance the effect caused by the corticosteroid as well.

Further studies by the applicant also show that even low therapeutic concentrations of 1,8-cineole can significantly inhibit the production of IL-1beta (Interleukin-1beta) compared to subtherapeutic concentrations of corticosteroids (e.g. especially dexamethasone), for which no significant inhibition is demonstrated, i.e. by combining 1,8-cineole with the corticosteroids in question (e.g. especially dexamethasone), significant inhibition can be demonstrated even for subtherapeutic, but of course also for therapeutic doses or concentrations of corticosteroids (e.g. especially dexamethasone) with a resulting intensification of the effect of the corticosteroids.

However, according to the applicant's in vitro studies, cineole, especially 1,8-cineole, alone also has a significant inhibitory effect on the production of cytokines, especially the cytokines IL-8 (interleukin-8) and TNF-alpha (tumor necrosis factor-alpha), in human monocytes of volunteers.

The applicant's studies thus show overall that the active ingredient cineole, especially 1,8-cineole, especially in the form of the pure substance, is capable of attenuating or warding off the relevant course of infection, especially preventing or attenuating hypercytokinemia, in the prophylactic and/or therapeutic treatment of viral diseases caused by corona viruses, especially COVID-19. This antiviral effect of 1,8-cineole in COVID-19 infections is completely unexpected and can even be further enhanced or increased by systemic administration of corticosteroids (e.g. especially dexamethasone), especially in a synergistic manner.

Furthermore, as the applicant has equally surprisingly discovered, the cineole used according to the invention, especially 1,8-cineole, also acts as an antioxidant and NO-regulator with respect to oxidation methods and NO-deficiency states occurring in the body or organs of the affected patients in viral diseases caused by corona viruses, especially COVID-19.

Therefore, in the context of the use according to the invention, the cineole, especially 1,8-cineole, can also be used especially as an inducer of NO-production for or in the context of the treatment of viral diseases caused by corona viruses, especially COVID-19.

Furthermore, the cineole, especially 1,8-cineole, can also be used in the context of the use according to the invention, especially to improve tissue and/or microperfusion in NO-deficiency situations with respect to the organs concerned.

Because NO as such is basically known as an anti-inflammatory mediator as well as an inhibitor of inflammatory mediators and platelet aggregation and as an activator of ciliary function and mucosal clearance and protects especially against respiratory infections and exacerbations of respiratory infections. In this respect, cineole, especially 1,8-cineole, is therefore suitable as an active substance or therapeutic agent which normalizes the suppressed NO-production present in coronavirus infections by favorable degradation of O₂⁻-radicals with induction of NO and adequately adapts it to the respective requirements by modulation. This results in the context of the present invention in a completely new indication for the use of cineole, especially 1,8-cineole (e.g. preferably in a higher, systemically effective daily dose, for example, of e.g. 600 to 1,200 mg, especially for the regulation of organ perfusion and, especially, also for the protection of upper and lower airways, including the lungs).

In the context of the present invention, in the search for the underlying properties of systemically administered cineole, especially 1,8-cineole, especially to intensify anti-inflammatory effects, moreover, completely surprisingly, an antioxidant effect was also found for cineole, especially 1,8-cineole, especially as a result of inhibition of the production of superoxides (O₂⁻-radicals), the activity of superoxide dismutases (SOD) and of hydrogen peroxide (H₂O₂), which as an end product of oxidation stimulates the production of inflammatory mediators, especially cytokines and arachidonic acid metabolites. Here, surprisingly, an inhibition of the spontaneous production of O₂⁻-radicals at therapeutic concentrations of cineole, especially 1,8-cineole, was also demonstrated, whereby cineole, especially 1,8-cineole, already inhibits the production of O₂⁻-radicals and H₂O₂ at lower concentrations in the therapeutic range. Surprisingly, the cause of these antioxidant effects of cineole, especially 1,8-cineole, was found to be the action of cineole, especially 1,8-cineole, as an active inducer of NO-production. Thus, cineole, especially 1,8-cineole, was found to actively induce NO-production by mediating antioxidant effects. Thus, modulating influences of cineole, especially 1,8-cineole, to control oxidative and thus cell-damaging as well as proinflammatory influences by inhibiting O₂⁻-radicals and an opposite stimulation of anti-inflammatory NO in the therapeutic dose range of cineole, especially 1,8-cineole, could be demonstrated by the applicant.

Overall, cineole, preferentially 1,8-cineole, is thus efficiently suitable for use in the prophylactic and/or therapeutic treatment of viral diseases caused by corona viruses, especially COVID-19, based on the surprisingly discovered findings of the applicant (e.g. as a sole therapeutic agent or else in co-therapy with further active ingredients, as described hereinabove, especially corticosteroids, such as dexamethasone).

As previously stated, it is an object of the present invention—according to a first aspect of the invention—to provide a cineole, preferentially 1,8-cineole, for use in the prophylactic and/or therapeutic treatment of viral diseases caused by corona viruses, especially COVID-19 (i.e. in other words, the use of a cineole, preferentially 1,8-cineole, for the prophylactic and/or therapeutic treatment of viral diseases caused by corona viruses, especially COVID-19).

According to a particular embodiment of the present invention in accordance with this aspect of the invention, it is provided that the cineole, preferentially 1,8-cineole, is applied systemically, especially perorally or parenterally, preferentially perorally, and/or that the cineole, preferentially 1,8-cineole, is prepared for systemic, especially peroral or parenteral, preferentially peroral, application. In this way, high systemic active doses or active ingredient levels (i.e. active ingredient levels or concentrations) are achieved, so that particularly good efficacy or efficiency can be realized.

According to an advantageous embodiment according of this aspect of the invention, it is particularly provided that the cineole, preferentially 1,8-cineole, is applied in the form of a dosage form to be administered perorally and/or that the cineole, preferentially 1,8-cineole, is prepared in a dosage form to be administered perorally.

According to another particular embodiment of the present invention according to this aspect, it may be particularly provided that the cineole, preferentially 1,8-cineole, is administered as an entericcoated (gastric juice resistant) but small intestine soluble systemic dosage form, preferably as a capsule, dragee, pill, tablet or the like, and/or that the cineole, preferentially 1,8-cineole, is prepared for administration as an entericcoated (gastricjuice resistant) but small intestine soluble systemic dosage form, preferably a capsule, dragee, pill, tablet or the like.

For the systemic application of cineole, especially 1,8-cineole, various preparations are commercially available, especially based on generally entericcoated (gastric juice resistant) but small intestine-soluble dosage forms or capsules.

The advantage of such dosage forms is that, on the one hand, the active ingredient cineole, especially 1,8-cineole, is present with long-term stability and, on the other hand, particularly high systemic active ingredient concentrations or active ingredient levels can be achieved. In this way, particularly high-dose dosage forms can also be provided, so that precise and high dosing of the active ingredient is possible.

In the context of the present invention, the cineole, especially 1,8-cineole, is usually used in a peroral dosage form, preferentially in the form of capsules. According to the invention, it is particularly preferred to apply the cineole, especially the 1,8-cineole, in the form of a peroral administration, especially a capsule, which is resistant to gastric juice but soluble in the small intestine. Peroral, entericcoated (gastric juice resistant), but small intestine-soluble dosage forms, especially capsules, which contain cineole, especially 1,8-cineole, as a pure substance are particularly preferred. Such products are commercially available (e.g. Soledum® capsules, distributed by Cassella-med GmbH & Co. KG and Maria Clementine Martin Klosterfrau Vertriebsgesellschaft mbH, both Cologne, Germany).

In general, it is provided in the context of the present invention that the peroral dosage form, especially capsule, is designed to be entericcoated (gastric juice resistant) but soluble in the small intestine. This achieves a particularly optimal release profile since the active ingredient is released in a targeted and purposeful manner only in the intestine.

The definition of the terms “entericcoated” (gastric juice resistant) and “small intestine-soluble” used in the context of the present invention, as well as the related test methods, are given in European Pharmacopoeia 7.0, 04/2010: 1502, pages 707 to 709, keyword “Capsules”, subchapter “Gastro-Resistant Capsules”, and European Pharmacopoeia 7.1, 04/2011: 20901, pages 3331 and 3332, keyword “2.9.1 Disintegration of Tablets and Capsules”.

In the context of the present invention, the term “entericcoated” (gastric juice resistant) is to be especially understood as meaning that the capsules can be kept with constant mixing, especially stirred, for at least two hours in 0.1 N hydrochloric acid, which is heated to temperatures of 35 to 39° C., without the capsules showing signs of decomposition or cracking or other damage.

On the other hand, in the context of the present invention, the term “soluble in the small intestine” especially means that the capsules are decomposed in an aqueous phosphate buffer solution, which is adjusted to a pH of about 6.8, with stirring at temperatures in the range of 35 to 39° C. within one hour to such an extent that the active substance is released.

In the context of the present invention, it is envisaged that the cineole, preferentially 1,8-cineole, is administered in pharmaceutically effective or therapeutically effective amounts and/or that the cineole, preferentially 1,8-cineole, is prepared for administration in pharmaceutically effective or therapeutically effective amounts.

Especially, it may be provided in the context of the present invention according to this aspect of the invention according to a particular embodiment that the cineole, preferentially 1,8-cineole, is administered at a daily dose in the range of from 1 to 5,000 mg/diem, especially in the range of from 2 to 3,000 mg/diem, preferentially in the range of from 5 to 2,500 mg/diem, preferably in the range of from 10 to 2,000 mg/diem, more preferably in the range of from 50 to 1. 500 mg/diem, and/or that the cineole, preferentially 1,8-cineole, is prepared for administration at a daily dose in the range of from 1 to 5,000 mg/diem, especially in the range of from 2 to 3,000 mg/diem, preferentially in the range of from 5 to 2,500 mg/diem, preferably in the range of from 10 to 2,000 mg/diem, more preferably in the range of from 50 to 1,500 mg/diem.

According to a further particular embodiment of the present invention according to this aspect of the invention, it may especially be provided that the cineole, preferentially 1,8-cineole, is present and/or administered as a pure substance. Especially, the cineole, preferentially 1,8-cineole, may thereby be present and/or administered with a purity of at least 95 wt. %, especially at least 96 wt. %, preferably at least 97 wt. %, more preferably at least 98 wt. %, even more preferably at least 99 wt. %, most preferably at least 99.5 wt. %, based on the cineole, preferentially 1,8-cineole. Especially, it is preferred in this context according to the invention that the cineole, preferentially 1,8-cineole, is free of other terpenes and/or that the cineole, preferentially 1,8-cineole, does not contain other terpenes.

In other words, in the context of the present invention according to this aspect of the invention, according to a particular embodiment, it may especially be provided that the cineole, preferentially 1,8-cineole, is present and/or administered in the absence of other (further) terpenes.

According to a particular embodiment according to the invention, it may thus be provided especially that the cineole, preferentially 1,8-cineole, is present and/or administered as a pure substance and/or in the absence of other (further) terpenes.

As described above, it is therefore preferred in accordance with the invention that the monoterpene-based active substance (i.e. cineole, preferentially 1,8-cineole) is used in the form of a pure substance, i.e. as the only or technically isolated/purified active substance. In this way, for example, an always constant dosage or an always constant content of active substance can be ensured in the dosage form provided according to the invention. High systemic active ingredient doses or levels can also be achieved in this way.

Above all, as explained above, cineole, especially 1,8-cineole, in its pure form or as a pure substance has a particularly efficient steroid-like action potential, since in its pure form or as a pure substance it causes a particularly efficient inhibition of inflammatory mediators or of leukotriene and cytokine production. In contrast, mixed essential oils containing cineole as one of many active components (such as eucalyptus oil etc.) stimulate prostaglandin production and show only a significantly reduced inhibition of leukotriene and cytokine production compared to pure cineole, since the mixed oils also contain substances that can stimulate cell activity and mediator production and therefore do not have an anti-inflammatory effect, but rather can cause intolerance reactions (i.e. such mixed essential oils or oil mixtures can consequently generally increase cell activity and even induce inflammatory mediator production). In contrast, however, isolated or pure cineole, especially 1,8-cineole, exclusively causes a significant inhibition of inflammatory mediator production.

It is particularly preferred that the cineole, especially 1,8-cineole, is administered in pure form or as a pure substance and systemically. This is because the cineole, especially 1,8-cineole, in pure form or as a pure substance is absorbed into the blood after systemic administration, especially in the form of capsules that are entericcoated (gastric juice resistant) but soluble in the small intestine and is released into the alveoli in accordance with its physical properties, even in high active ingredient doses or concentrations. As a result, cineole, especially 1,8-cineole, can have an anti-inflammatory effect in the smallest, peripherally located respiratory tracts as part of a therapy for viral infections or viral diseases caused by corona viruses, especially COVID-19.

According to yet another particular embodiment according to this aspect of the invention, it may be provided according to the invention that the cineole, preferentially 1,8-cineole, is present and/or administered together with at least one physiologically acceptable carrier (excipient). Especially, it may be provided that the physiologically acceptable carrier (excipient) is miscible with and/or soluble therein. Furthermore, it may especially be provided that the physiologically acceptable carrier (excipient) is present in liquid or solid, preferably liquid, aggregate state at 20° C. and at atmospheric pressure. In this context, it may be further provided that the physiologically acceptable carrier (excipient) may be selected from the group of fatty acid esters, preferably triglycerides of fatty acids, more preferably medium chain triglycerides (MCT), even more preferably triglycerides of C₆-C₁₂-fatty acids.

According to a particular embodiment, the dosage form provided according to the invention thus contains the monoterpene and active ingredient cineole, especially 1,8-cineole, together with at least one physiologically harmless carrier (excipient) which is miscible with the active substance and/or soluble therein, especially liquid at 20° C. and atmospheric pressure. The carrier or excipient should not itself be pharmacologically active but should form a preferably homogeneous mixture or solution with the active substance.

According to a preferred embodiment of the invention, the carrier or excipient can be selected from the group of fatty acid esters, preferably triglycerides of fatty acids, more preferably medium chain triglycerides (MCT), even more preferably triglycerides of C₆-C₁₂-fatty acids.

Especially, mixtures of fatty acid triglycerides which are liquid at room temperature (especially 20° C.) and atmospheric pressure can be preferably used as carriers or excipients; especially, this term refers to esters of the trivalent alcohol glycerol (propane-1,2-3-triol) with three, usually different, predominantly even-numbered and unbranched aliphatic monocarboxylic acids, the so-called fatty acids. Compounds of this type are also called triglycerides (according to IUPAC recommendation: triacylglycerols). Triglycerides, also known synonymously as glycerol triester, are thus triple esters of the trivalent alcohol glycerol with three acid molecules, the prefix “tri” referring to three acyl acid residues esterified with glycerol.

Especially, medium-chain triglycerides, as are preferably used according to the invention as a carrier or excipient for the monoterpene-containing active substance, are especially semisynthetic neutral glycerol esters of saturated, generally unbranched monocarboxylic acids of medium chain length (i.e. C₆-C₁₂-chains). Especially, the term medium-chain triglycerides refers to mixtures of triglycerides of saturated fatty acids, mainly caprylic acid (octanoic acid) and capric acid (decanoic acid). Medium-chain triglycerides can generally be produced from oil extracted from the solid and dried part of the endosperm of Cocos nucifera L. and/or from the dried endosperm of Elaeis guineenses Jacq. For further details on the concept of medium-chain triglycerides, reference can be made, for example, to the Ph. Eur. monograph, 6th edition, basic work 2008, pages 4224 to 4226, as well as to the Zeitschrift für Emährungswissenschaft, volume 13, issue 1/2, 1973, pages 6 ff, D. Sailer et al. “Mittelkettige Triglyceride—Klinische Physiologie und Anwendung”).

According to the invention, it is preferred if the carrier or excipient is used in an active substance/carrier quantitative ratio in the range of from 1,000:1 to 1:1,000, especially 100:1 to 1:100, preferentially 50:1 to 1:50, more preferably 10:1 to 1:10, even more preferably 5:1 to 1:2, most preferably 3:1 to 1:1. By mixing the active substance (i.e. cineole, especially 1,8-cineole) with the excipient, a significantly improved compatibility of the active substance with the other ingredients of the dosage form and an improved stability, especially storage stability, are achieved.

According to an equally further particular embodiment of the present invention, it may be provided that the cineole, preferentially 1,8-cineole, is present and/or administered in the form of a composition, especially a pharmaceutical composition, especially together with at least one pharmaceutically acceptable and/or physiologically acceptable excipient (carrier).

In this context, it may be particularly envisaged that the composition contains the cineole, preferentially 1,8-cineole, as the only active ingredient, especially as the only pharmaceutical active ingredient.

Furthermore, it may be provided especially that the composition contains the cineole, preferentially 1,8-cineole, as a pure substance, preferentially with a purity of at least 95 wt. %, especially at least 96 wt. %, preferably at least 97 wt. %, more preferably at least 98 wt. %, even more preferably at least 99 wt. %, most preferably at least 99.5 wt. %, based on the cineole, preferentially 1,8-cineole, and/or preferentially free of other terpenes.

Similarly, it may be provided in this context that the composition contains the cineole, preferentially 1,8-cineole, free of other terpenes and/or that the composition does not contain any other terpene and/or that the composition is free of terpenes other than cineole, preferentially 1,8-cineole.

Furthermore, it may be provided in this context that the composition contains the cineole, preferentially 1,8-cineole, in effective, especially pharmaceutically and/or therapeutically effective, amounts.

Likewise, in this context, it may be provided according to the invention that the composition contains the cineole, preferentially 1,8-cineole, in relative amounts, based on the composition, in the range of from 0.0001 to 80 wt. %, especially of from 0.001 to 75 wt. %, preferentially of from 0.005 to 70 wt. %, preferably of from 0.01 to 60 wt. %, more preferably of from 0.05 to 55 wt. %, even more preferably of from 0.1 to 50 wt. %.

Finally, in accordance with the invention, it may also be provided in this context that the at least one pharmaceutically acceptable and/or physiologically acceptable excipient (carrier) is miscible with 1,8-cineole and/or soluble therein. Preferentially, the at least one pharmaceutically acceptable and/or physiologically acceptable excipient (carrier) may be in liquid or solid aggregate state at 20° C. and at atmospheric pressure. Also preferably, the at least one pharmaceutically acceptable and/or physiologically acceptable excipient (carrier) can be selected from the group of fatty acid esters, preferably triglycerides of fatty acids, more preferably medium chain triglycerides (MCTs), even more preferably triglycerides of C₆-C₁₂-fatty acids.

According to a particular embodiment of the present invention, the cineole, preferentially 1,8-cineole, may be present and/or administered in liposome-encapsulated and/or liposome-packaged form. This ensures a particularly good release profile.

According to another particular embodiment of the present invention, the cineole, preferentially 1,8-cineole, may be present and/or administered in micellar form and/or in a micellar dosage form. This can also ensure a particularly good release profile.

Furthermore, according to a particular embodiment, it may be provided that the cineole is present and/or used and/or administered without and/or in the absence of non-steroidal anti-inflammatory drugs (NSAID).

According to another particular embodiment of the present invention in accordance with this aspect of the invention, it may be provided that the cineole, preferentially 1,8-cineole, is used or applied together with at least one further active ingredient. In this context, it may be envisaged especially that the further active ingredient is selected from (i) anti-inflammatory active ingredients, especially corticosteroids, (ii) blood-thinning or blood-clotting active ingredients, (iii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof.

According to a particularly preferred embodiment, it may be provided especially that the further active ingredient is used and/or administered separately, especially spatially separated, from the cineole, preferentially 1,8-cineole, but functionally coherent therewith, especially in the form of a kit (kit-of-part)

In the aforementioned manner (i.e. combination or co-administration with at least one further active ingredient), it is especially possible to significantly increase the effect or efficiency of the further active ingredient, especially in the case of corticosteroids (such as e.g. dexamethasone), especially in a synergistic manner, and to significantly reduce the dose quantities used thereof, associated with the associated advantages (e.g. avoidance or reduction of side effects etc.). At the same time, the interaction can significantly increase the sensitivity to the additional active ingredient, especially in the case of corticosteroids (such as dexamethasone). This increase in effect can in turn possibly—without wishing to commit to a specific theory—probably be explained by the steroid-like effect potential of the systemically used cineole, especially 1,8-cineole, which was surprisingly discovered by the applicant, especially with regard to the inhibition of inflammatory mediators.

As previously stated, it may be provided in the context of the present invention that the cineole, preferentially 1,8-cineole, is used as a co-therapeutic agent and/or as a co-medication in the context of a COVID-19 basic therapy and/or that the cineole, preferentially 1,8-cineole, is used as or in combination therapy to a COVID-19 basic therapy and/or existing COVID-19 therapy. As previously stated, the cineole, preferentially 1,8-cineole, used according to the invention can lead to a significant, especially synergistic, increase in the effect of the basic therapeutic agent(s), such as especially corticosteroids (e.g. dexamethasone).

According to a further particular embodiment of the present invention, it may be envisaged that the cineole, preferentially 1,8-cineole, is used for prophylactic and/or therapeutic treatment, especially for suppression or attenuation, of a hyperinflammatory syndrome occurring in the context of a COVID-19 infection, optionally in co-medication with a preferably systemically applied corticosteroid, especially dexamethasone.

Furthermore, according to a particular embodiment of the present invention, it may be provided that the cineole, preferentially 1,8-cineole, exerts suppressing or attenuating effects with respect to a hyperinflammation syndrome occurring in the context of a COVID-19 infection, optionally in co-medication with a preferably systemically applied corticosteroid, especially dexamethasone.

Furthermore, it may be provided in the context of the present invention according to a again further particular embodiment that the cineole, preferentially 1,8-cineole, is used for prophylactic and/or therapeutic treatment, especially for suppression or attenuation, of a hypercytokinemia (cytokine storm) occurring in the context of a COVID-19 infection, especially of a cytokine release syndrome (CRS), optionally in co-medication with a preferably systemically applied corticosteroid, especially dexamethasone.

According to yet another particular embodiment of the present invention, it may be provided according to the invention that the cineole, preferentially 1,8-cineole, exerts suppressing or attenuating effects with respect to a hypercytokinemia (cytokine storm) occurring in the context of a COVID-19 infection, especially with respect to a cytokine release syndrome (CRS), optionally in co-medication with a preferably systemically applied corticosteroid, especially dexamethasone.

As stated above, it may be particularly envisaged in the context of the present invention that the cineole, preferentially 1,8-cineole, is used to suppress or attenuate inflammatory mediation occurring in the context of a COVID-19 infection, especially hypercytokinemia (cytokine storm) occurring in the context of a COVID-19 infection.

Likewise, it may be particularly provided in the context of the present invention that the cineole, especially 1,8-cineole, exerts suppressive or attenuating action with respect to inflammatory mediation occurring in the context of a COVID-19 infection, especially with respect to hypercytokinemia (cytokine storm) occurring in the context of a COVID-19 infection.

Furthermore, according to another particular embodiment of the present invention, it may be provided that the cineole, preferentially 1,8-cineole, is used to suppress the replication of corona viruses, especially for suppression of the replication of corona viruses occurring in thrombocytes, especially in the context of COVID-19 infection.

Especially, it may be provided in the context of the present invention that the cineole, preferentially 1,8-cineole, exerts suppressive action with respect to replication of corona viruses, especially with respect to replication of corona viruses taking place in thrombocytes, especially in the context of a COVID-19 infection. This aspect has already been explained and presented at the beginning.

Furthermore, it can be provided within the scope of the present invention according to a further particular embodiment that the cineole, preferentially 1,8-cineole, is used for the prophylactic and/or therapeutic treatment of systemic organ involvement (organ diseases) occurring in viral diseases caused by corona viruses, especially COVID-19, especially bronchopulmonary, cardiac and/or renal organ involvement (organ diseases), and/or of neurological symptoms, complications or manifestations (e.g. loss or disturbances of the sense of smell) occurring in viral diseases caused by corona viruses, especially COVID-19. As described at the beginning, the anti-inflammatory, especially the cytokine production inhibiting effect of cineole, especially 1,8-cineole, is not organ-specific or not site-specific, so that cineole, especially 1,8-cineole, is particularly suitable for this specific application or use.

As also set forth previously, it may be equally provided in the context of the present invention that the cineole, preferentially 1,8-cineole, is used and/or functions as an inducer of NO-production and/or for remedying or alleviating NO-deficiency states (NO-deficiency situations) occurring in the body of a diseased patient in viral diseases caused by corona viruses, especially COVID-19. In this way, the viral disease, especially COVID-19, is causally counteracted.

Finally, according to a particular embodiment of the present invention—as also explained and described above—it may be equally provided that the cineole, preferentially 1,8-cineole, is used and/or functions for remedying or alleviating oxidative and/or inflammatory conditions in the body of a diseased patient occurring in viral diseases caused by corona viruses, especially COVID-19. Also in this way, an antiviral therapy of the viral disease is effected or realized.

The present invention, both according to the first aspect of the present invention and according to all other aspects of the present invention, is thus associated with a plurality of advantages and special features which make the therapy concept according to the invention unique and special, especially highly efficient.

A further subject matter of the present invention—according to a second aspect of the present invention—is the inventive use of cineole, especially 1,8-cineole, for the prophylactic and/or therapeutic treatment of viral diseases caused by corona viruses, especially COVID-19, or the inventive use of a cineole, preferentially 1,8-cineole, for producing a drug or medicament for the prophylactic and/or therapeutic treatment of viral diseases caused by corona viruses, especially COVID-19.

In the context of the use according to the present aspect of the invention, it is particularly intended that the active ingredient or the cineole, preferentially 1,8-cineole, is applied systemically.

Furthermore, within the scope of the use according to the invention according to this aspect of the invention, it may be provided, if necessary, that the active ingredient or the cineole, preferentially 1,8-cineole, is used or applied together with at least one further active ingredient. The further active ingredient may especially be selected from (i) anti-inflammatory active ingredients, especially corticosteroids, (ii) blood-thinning or blood-clotting active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof. Especially, the additional active ingredient is equally systemically applied or administered.

For further details concerning this aspect of the invention, reference may be made to the foregoing explanations concerning the first aspect of the invention, which explanations apply equally to the present aspect of the invention.

Similarly, a subject matter of the present invention—according to a third aspect of the present invention—is the inventive use of a cineole, preferentially 1,8-cineole, as an antiviral agent for the prophylactic and/or therapeutic treatment of viral diseases caused by corona viruses, especially COVID-19, or the inventive use of a cineole, preferentially 1,8-cineole, for producing an antiviral agent for the prophylactic and/or therapeutic treatment of viral diseases caused by corona viruses, especially COVID-19.

In the context of the use according to the present aspect of the invention, it is particularly intended that the active ingredient or the cineole, preferentially 1,8-cineole, is applied systemically.

Furthermore, within the scope of the use according to the invention according to this aspect of the invention, it may be provided, if necessary, that the active ingredient or the cineole, preferentially 1,8-cineole, is used or applied together with at least one further active ingredient. The further active ingredient may especially be selected from (i) anti-inflammatory active ingredients, especially corticosteroids, (ii) blood-thinning or blood-clotting active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof. Especially, the additional active ingredient is equally systemically applied or administered.

For further details on this aspect of the invention, reference may be made to the above discussion of the aspects of the invention previously described, which discussion applies equally to the present aspect of the invention.

Yet another subject matter of the present invention—according to a fourth aspect of the present invention—is an inventive method for the prophylactic and/or therapeutic treatment of viral diseases caused by corona viruses, especially COVID-19, wherein in the method a pharmaceutically effective or therapeutically effective amount of a cineole, especially 1,8-cineole, is administered to a patient suffering from a viral disease caused by corona viruses, especially COVID-19.

Within the scope of the method according to the present aspect of the invention, it is particularly provided that the active substance or the cineole, preferentially 1,8-cineole, is applied systemically.

Furthermore, within the scope of the method according to the present aspect of the invention, it may be provided, if necessary, that the active ingredient or the cineole, preferentially 1,8-cineole, is used or applied together with at least one further active ingredient. The further active ingredient may especially be selected from (i) anti-inflammatory active ingredients, especially corticosteroids, (ii) blood-thinning or blood-clotting active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof. Especially, the additional active ingredient is equally systemically applied or administered.

For further details on this aspect of the invention, reference may be made to the above discussion of the aspects of the invention previously described, which discussion applies equally to the present aspect of the invention.

Also a subject matter of the present invention—according to a fifth aspect of the present invention—is a drug or medicament, especially an antiviral agent, for (use in the) prophylactic and/or therapeutic treatment of viral diseases caused by corona viruses, especially COVID-19, wherein the drug or medicament, especially antiviral agent, contains a cineole, especially 1,8-cineole, together with at least one pharmaceutically acceptable and/or physiologically harmless excipient (carrier).

Also within the scope of this aspect of the invention, it is particularly intended that the active ingredient or the cineole, preferentially 1,8-cineole, or the drug or medicament, especially antiviral agent, is applied systemically.

Furthermore, it may also be provided within the scope of this aspect of the invention, if necessary, that the active ingredient or the cineole, preferentially 1,8-cineole, or the drug or medicament, especially antiviral agent, is used or applied together with at least one further active ingredient. The further active ingredient may especially be selected from (i) anti-inflammatory active ingredients, especially corticosteroids, (ii) blood-thinning or blood-clotting active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof. Especially, the additional active ingredient is equally systemically applied or administered.

According to the invention, it is preferred if the inventive drug or medicament contains the cineole, especially 1,8-cineole, as the only active ingredient, especially as the only pharmaceutical active ingredient.

According to a particular embodiment according to this aspect of the invention, the inventive drug or medicament may contain the cineole, preferentially 1,8-cineole, especially as a pure substance, preferentially with a purity of at least 95 wt. %, especially at least 96 wt. %, preferably at least 97 wt. %, more preferably at least 98 wt. %, even more preferably at least 99 wt. %, most preferably at least 99.5 wt. %, based on the cineole, preferentially 1,8-cineole, and/or preferentially free of other terpenes.

According to a further particular embodiment according to this aspect of the invention, it may be provided that the inventive the drug or medicament contains the cineole, preferentially 1,8-cineole, free of other terpenes; and/or that the drug or medicament does not contain any other terpene; and/or that the inventive drug or medicament is free of terpenes other than cineole, preferentially 1,8-cineole.

According to yet another particular embodiment according to this aspect of the invention, it may be provided that the inventive drug or medicament contains the cineole, especially 1,8-cineole, in effective, especially pharmaceutically and/or therapeutically effective, amounts.

Especially, it may be provided in this context that the inventive drug or medicament contains the cineole, preferentially 1,8-cineole, in relative amounts in the range of from 0.0001 to 80 wt. %, especially in the range of from 0.001 to 75 wt. %, preferentially in the range of from 0.005 to 70 wt. %, preferably in the range of from 0.01 to 60 wt. %, more preferably in the range of from 0.05 to 55 wt. %, even more preferably in the range of from 0.1 to 50 wt. % According to yet another particular embodiment according to this aspect of the invention, it may be provided that the at least one pharmaceutically acceptable and/or physiologically acceptable excipient (carrier) is miscible with 1,8-cineole and/or soluble therein. Preferentially, the at least one pharmaceutically acceptable and/or physiologically acceptable excipient (carrier) may be in the liquid or solid aggregate state at 20° C. and at atmospheric pressure. Also preferably, the at least one pharmaceutically acceptable and/or physiologically acceptable excipient (carrier) can be selected from the group of fatty acid esters, preferably triglycerides of fatty acids, more preferably medium-chain triglycerides (MCT), even more preferably triglycerides of C₆-C₁₂-fatty acids.

For further details on this aspect of the invention, reference may be made to the above discussion of the aspects of the invention previously described, which discussion applies equally to the present aspect of the invention.

Yet another subject matter of the present invention—according to a sixth aspect of the present invention—is a composition, especially a pharmaceutical composition, for (use in the) prophylactic and/or therapeutic treatment of viral diseases caused by corona viruses, especially COVID-19, wherein the composition contains a cineole, preferentially 1,8-cineole, especially together with at least one pharmaceutically acceptable and/or physiologically acceptable excipient (carrier).

Also within the scope of this aspect of the invention, it is particularly intended that the active ingredient or the cineole, preferentially 1,8-cineole, or the composition, especially pharmaceutical composition, is applied systemically.

Furthermore, it may also be provided within the scope of this aspect of the invention, if necessary, that the active ingredient or the cineole, preferentially 1,8-cineole, or the composition, especially pharmaceutical composition, is used or applied together with at least one further active ingredient. The further active ingredient may especially be selected from (i) anti-inflammatory active ingredients, especially corticosteroids, (ii) blood-thinning or blood-clotting active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof. Especially, the additional active ingredient is equally systemically applied or administered.

According to the invention, it is preferred if the inventive composition, especially pharmaceutical composition, contains the cineole, preferentially 1,8-cineole, as the only active ingredient, especially as the only pharmaceutical active ingredient.

According to a particular embodiment according to this aspect of the invention, the inventive composition, especially pharmaceutical composition, contains the cineole, preferentially 1,8-cineole, as a pure substance, preferentially with a purity of at least 95 wt. %, especially at least 96 wt. %, preferably at least 97 wt. %, more preferably at least 98 wt. %, even more preferably at least 99 wt. %, most preferably at least 99.5 wt. %, based on the cineole, preferentially 1,8-cineole, and/or preferentially free of other terpenes.

According to a further particular embodiment according to this aspect of the invention, it may be provided that the inventive composition, especially pharmaceutical compositions, contains the cineole, preferentially 1,8-cineole, free of other terpenes and/or that the inventive composition, especially pharmaceutical composition, does not contain any other terpene and/or that the inventive composition, especially pharmaceutical composition, is free of terpenes other than cineole, preferentially 1,8-cineole.

According to yet another particular embodiment according to this aspect of the invention, it may be provided that the inventive composition, especially pharmaceutical composition, contains the cineole, preferentially 1,8-cineole, in effective, especially pharmaceutically and/or therapeutically effective, amounts.

Especially, it may be provided in this context that the inventive composition contains the cineole, preferentially 1,8-cineole, in relative amounts, based on the composition, in the range of from 0.0001 to 80 wt. %, especially 0.001 to 75 wt. %, preferentially 0.005 to 70 wt. %, preferably 0.01 to 60 wt. %, more preferably 0.05 to 55 wt. %, even more preferably 0.1 to 50 wt. %.

According to yet another particular embodiment according to this aspect of the invention, it may be provided that the at least one pharmaceutically acceptable and/or physiologically acceptable excipient (carrier) is miscible with 1,8-cineole and/or soluble therein. Preferably, the at least one pharmaceutically acceptable and/or physiologically acceptable excipient (carrier) may be in the liquid or solid state at 20° C. and at atmospheric pressure. Also preferably, the at least one pharmaceutically acceptable and/or physiologically acceptable excipient (carrier) may be selected from the group of fatty acid esters, preferably triglycerides of fatty acids, more preferably medium-chain triglycerides or MCT), even more preferably triglycerides of C₆-C₁₂-fatty acids.

For further details on this aspect of the invention, reference may be made to the above discussion of the aspects of the invention previously described, which discussion applies equally to the present aspect of the invention.

Finally, it is equally a subject matter of the present invention—according to a seventh aspect of the present invention—to provide a pharmaceutical combination for (use in the) prophylactic and/or therapeutic treatment of viral diseases caused by corona viruses, especially COVID-19, wherein the pharmaceutical combination comprises at least the following components (A) and (B):
(A) a cineole, preferentially 1,8-cineole; and
(B) at least one further active ingredient selected from (i) anti-inflammatory active ingredients, especially corticosteroids, (ii) blood-thinning or anticoagulant active ingredients, (iiii) antiviral active ingredients, especially synthetic antiviral active ingredients, such as Remdesivir, or protein-based antiviral active ingredients, such as especially monoclonal antibodies, immunoglobulins and interferons; (iv) cardiovascular active ingredients, especially antihypertensives and ACE inhibitors; as well as combinations thereof.

Also within the scope of this aspect of the invention, it is particularly intended that the active ingredient of component (A) or the cineole, preferentially 1,8-cineole, is applied systemically.

Especially, the other active ingredient of component (B) can also be used or applied systemically in the same way.

According to a particular embodiment of this aspect of the invention, it may especially be provided that the components (A) and (B) are present and/or administered separately from one another, especially spatially separate from one another, but functionally coherent and/or functionally associated with each other.

Furthermore, according to another particular embodiment of this aspect of the invention, it may especially be provided that the pharmaceutical combination is in the form of a kit (kit-of-parts), especially as a kit of components (A) and (B), and/or that components (A) and (B) are present and/or prepared and/or administered as a kit (kit-of-parts).

In the context of the present invention, a kit or kit-of-parts is understood to especially mean a unit or arrangement or combination of the two components (A) and (B), in which the two components (A) and (B) are present separated and/or separately, especially spatially separated and/or spatially separately, but are provided or administered as functionally interrelated or as functionally mutually associated components.

For further details on this aspect of the invention, reference may be made to the above discussion of the aspects of the invention previously described, which discussion applies equally to the present aspect of the invention.

Further embodiments, modifications and variations as well as advantages of the present invention are readily apparent and realizable to those skilled in the art upon reading the description without departing from the scope of the present invention.

The following embodiments are merely illustrative of the present invention, but without limiting the present invention thereto.

### EXAMPLES

### Example 1: Studies and Observations Related to Thrombocytes and Inflammatory Mediation Associated with Cineole Therapy

As already explained at the beginning and as surprisingly found by the applicant in this context, thrombocytes (blood platelets), which in addition to blood clotting also function as immunoregulatory inflammatory cells, can harbor coronaviruses and thus trigger viral infections triggered by coronaviruses, especially COVID-19, whereby the active ingredient 1,8-cineole can especially counteract a hyperinflammation syndrome occurring in the context of the coronavirus infection and above all a hypercytokinemia (cytokine storm) and is capable of containing the coronavirus infection. In previous COVID-19 infection courses, it is known that especially two states of inflammation can occur, namely the one leading to hyperinflammation with a potentially critical course or the one leading to viral clearance. Above all, platelets are known from blood coagulation and the maintenance of vascular integrity; but recent studies also show that platelets are important active regulators of the immune system and are especially attributable to the innate immune system. In this context, the importance of platelets in inflammation-related diseases has been demonstrated, thus providing insights into pathomechanisms.

In the context of viral infections, platelets are particularly capable of inducing and maintaining massive inflammatory responses (especially hypercytokinemia) as well as presenting antigens and initiating immune responses in viral diseases, but also of taking up RNA and passing it horizontally to other cells, and furthermore of taking up RNA viruses and providing them with a replication site. Furthermore, platelets are able to produce so-called platelet-derived microvesicles or PMPs and regulate them through PMP immune cells. Based on clinical observations in COVID-19 infections, thrombocytes may be considered or evaluated as central target cells of COVID-19 infection: Thus, an altered platelet/lymphocyte ratio (so-called platelet-to-lymphocyte ratio or PTR) can be used as an inflammatory marker and prognostic factor with respect to the course of COVID-19 infections. Altered coagulation behavior also suggests that PTL inhibition is associated with a milder course in COVID-19 infections. Thrombocytopenia, on the other hand, may be considered a marker for a severe COVID-19 course. COVID-19 infections are generally characterized by a variety of proinflammatory effector cytokines released from platelets, such as TNF, IL1β, IL-6, IL-8, G-CSF, and GM-CSF.

In the context of the present experiments, a basic clinical and immunological understanding can be gained by the applicant with regard to the alteration of thrombocytes by 1,8-cineole in the context of COVID-19 infections under cineole treatment. In this context, it can be shown on the part of the applicant that cineole, especially 1,8-cineole, can be used efficiently in the treatment of viral diseases caused by corona viruses, especially COVID-19.

In in-vitro-studies with thrombocytes obtained from healthy volunteers, which are first incubated with 1,8-cineole and then infected with corona viruses, it can be shown that an outbreak of infection, especially hypercytokinemia, can be prevented or reduced.

In further in-vitro-studies with thrombocytes obtained from patients suffering from COVID-19 incubated with 1,8-cineole, these results can be confirmed, especially to the effect that the course of infection, especially hypercytokinemia, can be prevented or attenuated. This antiviral effect of 1,8-cineole in COVID-19 infections is completely unexpected.

In this context, it is known that platelets adhere to lung epithelia and in this way enable or exacerbate infection of the epithelia with COVID-19. Adhesion to epithelia is in turn cytokine-dependent and consequently can be inhibited or reduced or attenuated by cineole, especially 1,8-cineole. This also mitigates or prevents infection of the epithelia and ultimately the lung tissue.

Analysis of the Alteration of Thrombocytes of COVID-19 Positive Patients

The alteration of thrombocytes in the three stages, i.e. initiation, full-form infection, and post-infection, will be studied, especially in the context of inflammation-mediated activation. Specifically, the following will be studied: the RNA texture, the proteome, the metabolome, the existence of platelet-derived microparticles.

Healthy thrombocytes are incubated with COVID-19 and functionally (i.e. cytokines) assayed. Subsequently, the virus can be inhibited both in its replication and in the production of the hyperinflammatory cytokines triggered in thrombocytes, which lead to the so-called cytokine storm, by incubation with 1,8-cineole.

In detail, in this context:
initially incubated healthy thrombocytes with 1,8-cineole, then infected with the virus to preemptively demonstrate show that infection can be prevented or reduced; and
COVID-19-infected thrombocytes incubated with 1,8-cineole show that cytokine storm can be prevented.

Thus, in the context of the present experiments, surprisingly, a fundamental clinical and immunological understanding can be gained by the applicant with respect to the alteration of thrombocytes in the context of COVID-19 infections under cineole treatment. In this context, it can be shown on the part of the applicant that cineole, especially 1,8-cineole, can be used efficiently in the treatment of viral diseases caused by corona viruses, especially COVID-19.

### Example 2: Studies and Observations Related to the Inhibition of the Production of Inflammatory Mediators, Especially Cytokines, by Cineole

As also explained at the beginning and as equally surprisingly found by the applicant in this context, the active ingredient 1,8-cineole equally causes an inhibition with regard to the production of inflammatory mediators, especially cytokines, and can thus counteract hypercytokinemia (cytokine storm) in the context of COVID-19 therapy.

In this context, cineole, especially 1,8-cineole, especially in its pure form, causes a significant inhibition of mediator production; this thus produces an anti-inflammatory effect. This effect is achieved with systemic application of cineole, especially 1,8-cineole, in the entire body and especially in the entire respiratory tract, especially in the entire lung, i.e. also as far as the periphery and the alveoli. This effect is significantly less pronounced with mixed essential oils.

Cineole, especially 1,8-cineole, especially in pure form, causes a significant inhibition of mediator production in this context: even low therapeutic concentrations of 1,8-cineole can already significantly inhibit the production of IL-1beta (interleukin-1beta) compared to subtherapeutic concentrations of corticosteroids (e.g. especially dexamethasone), for which no significant inhibition is demonstrated, i.e. by combining 1,8-cineole with the relevant corticosteroids (e.g. especially dexamethasone), nevertheless a significant inhibition can be achieved even for subtherapeutic but, of course, also for therapeutic concentrations of IL-1beta. i.e. by combining 1,8-cineole with the relevant corticosteroids (e.g. especially dexamethasone), with a resulting intensification of the effect of the corticosteroids.

Table 1 below shows the inhibitory activity of 1,8-cineole as pure compound (10⁻⁶ mol/l) on the one hand and dexamethasone on the other hand as well as their combinations with respect to LPS-stimulated production of IL-1beta in human monocytes in vitro. Monocyte IL-1beta production (n=14.4 experiments) in monocytes is significantly inhibited by cineole (10⁻⁶ mol/l) compared with the control. Mixed oil (eucalyptus oil) containing only about 60% 1,8-cineole at the same concentration (10⁻⁶ mol/l) inhibits IL-1beta production significantly weaker than 1,8-cineole as pure substance. Dexamethasone at subtherapeutic doses (10⁻⁹ mol/l) does not cause significant inhibition. In contrast, cineole and dexamethasone in combination synergistically inhibit IL-1beta production, i.e. more strongly than dexamethasone alone (and even at subtherapeutic concentrations of dexamethasone as low as 10⁻⁹ mol/l). Compared with cineole (10⁻⁶ mol/l) alone, IL-1beta production is synergistically and significantly more strongly inhibited by addition of dexamethasone (p<0.05).

The results from human monocyte cultures presented in Table 1 show that LPS-stimulated production of IL-1beta is significantly inhibited by 1,8-cineole and that LPS-stimulated production of IL-1beta is significantly more inhibited by a combination of 1,8-cineole and dexamethasone than by dexamethasone alone or even than by 1,8-cineole alone (see Table 1 below).

In this context, the experiments show an inhibitory effect with respect to the production of IL-1beta by the monoterpene 1,8-cineole on the one hand and by the corticosteroid dexamethasone on the other hand as well as their combination in LPS-stimulated human monocytes in vitro. The addition of even small amounts of 1,8-cineole causes a significant increase in the effect of the corticosteroid, accompanied by an increased inhibition of IL-1beta: monocytes (n=14.4 experiments) from healthy volunteers (10⁵/ml) are treated for 20 hours with 1,8-cineole (10⁻⁶ mol/I=0,015 μg/ml) and the corticosteroid dexamethasone (10⁻¹ mol/I or 10⁻¹ mol/l, respectively) and their combination are incubated in the presence of LPS (10 mg/ml). In cell culture supernatants, the production of IL-1beta is determined by ELISA (Cayman Chemical, Ann Arbor, Michigan, USA). 1,8-Cineole alone and therapeutically relevant concentrations of dexamethasone alone (10⁻¹ mol/l) each significantly inhibit IL-1beta production. In contrast, LPS-stimulated production is significantly more inhibited by a combination of 1,8-cineole (10⁻⁶ mol/l) plus dexamethasone than by dexamethasone alone. Especially, 1,8-cineole intensifies the effect of already subtherapeutic concentrations of dexamethasone.

TABLE 1
Inhibitory activity of 1,8-cineole (cineole), dexamethasone (dex.)
and combinations of 1,8-cineole and dexamethasone with respect
to LPS-stimulated production of IL-1beta in human monocytes

| concentration | IL-1beta | effect vs. control | |
|---|---|---|---|
| mol/l | pg/5 × 10⁻⁴ cells | inhibition (%) | p-value |
| control | 5252 ± 1017 | 0 ± 19 | — |
| mixing oil 10⁻⁶ | 4732 ± 473 | 9.9 ± 10 | 0.1901 |
| (eucalyptus oil) | | | |
| cineole 10⁻⁶ | 3548 ± 600 | 32.4 ± 17 | 0.0100 |
| dex. 10⁻¹² | 5245 ± 1027 | 0.1 ± 19 | 0.9634 |
| dex. 10⁻⁹ | 2655 ± 546 | 49.5 ± 20 | 0.0449 |
| cineole 10⁻⁶ + dex. 10⁻¹² | 3965 ± 641 | 24.6 ± 16 | 0.1904 |
| cineole 10⁻⁶ + dex. 10⁻⁹ | 348 ± 55 | 93.5 ± 16 | 0.0049 |

The applicant's studies thus show overall that the active ingredient cineole, especially 1,8-cineole, especially in the form of the pure substance, is capable of attenuating or warding off the relevant course of infection, especially preventing or attenuating hypercytokinemia, in the prophylactic and/or therapeutic treatment of viral diseases caused by corona viruses, especially COVID-19. This antiviral effect of 1,8-cineole in COVID-19 infections is completely unexpected and can even be further enhanced by systemic administration of corticosteroids (e.g. especially dexamethasone), especially in a synergistic manner.

### Example 3: Studies and Observations Related to an Antioxidant and NO-Regulatory Effect of Cineole

As equally stated at the outset and equally surprisingly found by the applicant, 1,8-cineole also acts as an antioxidant and NO-regulator with respect to oxidation methods and NO-deficiencies occurring in the body or organs of affected patients in viral diseases caused by corona viruses, especially COVID-19.

NO as such acts as an anti-inflammatory mediator as well as an inhibitor of inflammatory mediators and platelet aggregation and as an activator of ciliary function and mucosal clearance, protecting especially against respiratory infections and exacerbations of respiratory infections. In this respect, cineole, especially 1,8-cineole, is suitable as an agent or therapeutic agent to normalize the suppressed NO-production present in coronavirus infections by favorable degradation of O₂⁻-radicals with induction of NO and to adequately adapt it to the respective requirements by modulation.

In the present invention, modulatory antioxidant and anti-inflammatory effects of 1,8-cineole to control oxidative methods and induce nitric oxide (NO) production can be demonstrated. In search of the underlying property of 1,8-cineole to intensify anti-inflammatory effects in the therapy of COVID-19, an antioxidant effect for 1,8-cineole is found by inhibiting the production of superoxides (O₂⁻-radicals), the activity of superoxide dismutases (SOD) and hydrogen peroxide (H₂O₂). Here, inhibition of spontaneous and stimulated production of superoxides (O₂⁻-radicals) is demonstrated at therapeutic concentrations of 1,8-cineole, such that providing the substrate for dismutation of O₂⁻-radicals via superoxide dismutase activity partially inhibited by 1,8-cineole inhibits production of O₂⁻-radicals and H₂O₂ even at lower concentrations and in the therapeutic range. Surprisingly, the major cause of this antioxidant effect of 1,8-cineole is found to be its property as an active inducer of NO-production, i.e. 1,8-cineole is able to actively induce NO-production by mediating antioxidant effects (see Table 2 below).

Table 2 below shows the effect of 1,8-cineole on PMA-stimulated superoxide production (O₂⁻-production) (in RPMI-1640) of human monocytes in vitro. The dose-dependent effects of 1,8-cineole (4 experiments, n=14) on O₂⁻-production are measured by determination of INT formazan in culture supernatants (RPMI-1640) of human monocytes (10⁵/ml) after 20 hours. O₂⁻-production is not stimulated by PMA (500 mmol/l). For statistical analysis, the non-parametric Mann & Whitney test is used (p<0.05). Here, modulatory influences of 1,8-cineole to control oxidative and thus cell-damaging and proinflammatory influences (as they also occur with COVID-19) can be demonstrated by inhibition of O₂⁻-radicals and by an opposite stimulation of anti-inflammatory NO in the therapeutic range of 1,8-cineole.

TABLE 2
Effect of 1,8-cineole on PMA-stimulated superoxide production
(O₂⁻-production) in RPMI 1640 human monocytes in vitro

| 1,8-cineole | | INT-formazan | comparison to control | |
|---|---|---|---|---|
| μg/ml (mol/l) | | (nmol/10⁵) | (%) | p-value |
| spontaneous | | 4828 ± 251 | — | — |
| PMA | | 4203 ± 267 | −12.9 ± 6 | 0.0990 |
| 0.0015 | (10⁻⁸) | 5364 ± 229 | +27.6 ± 4 | 0.0083 |
| 1.2 | (8 × 10⁻⁶) | 4099 ± 234 | −2.5 ± 6 | 0.7721 |
| 1.5 | (10⁻⁵) | 2438 ± 389 | −42 ± 16 | 0.0024 |
| 3.0 | (2 × 10⁻⁵) | 634 ± 139 | −84.9 ± 22 | <0.0001 |

Thus, the above table 2 shows the concentration-dependent modulating effect of 1,8-cineole on O₂⁻—and NO-production in stimulated human monocytes in vitro: After stimulation (20 hours) of normal human monocytes (10⁵/ml), the production of NO is induced and of O₂⁻ suppressed in the control (i.e. without 1,8-cineole). In contrast, low subtherapeutic concentrations of 1,8-cineole slightly induce O₂⁻-production but already inhibit NO-production. In contrast, in the therapeutic dose range of 1,8-cineole, O₂⁻-production is strongly inhibited in the presence of strong or significant stimulatory effects on NO-production.

These results are also of integral importance for the understanding in relation to the therapy of coronavirus infections, especially COVID-19, with 1,8-cineole: Thus, an increased production of O₂⁻-radicals, as occurs in coronavirus infections, especially COVID-19, is permanently inhibited by therapy with 1,8-cineole and, moreover, is advantageously used as a substrate for the production of NO. NO, in turn, acts as an anti-inflammatory mediator as well as an inhibitor of inflammatory mediators and platelet aggregation as well as an activator of ciliary function and mucosal clearance and, in summary, protects against respiratory infections and their exacerbations. In this regard, 1,8-cineole is suitable as a COVID-19 therapeutic agent to normalize NO-production, which is suppressed in COVID-19, by favorable degradation of O₂⁻-radicals with induction of NO and to adequately adapt it to the respective requirements by modulation.

Overall, cineole, preferably 1,8-cineole, is thus efficiently suitable for use in the prophylactic and/or therapeutic treatment of viral diseases caused by corona viruses, especially COVID-19, on the basis of the surprisingly discovered findings of the applicant (e.g. as a sole therapeutic agent or, however, in co-therapy with further active ingredients, as previously described, especially corticosteroids, such as dexamethasone).

### Example 4: Further Studies and Observations Related to the Inhibition of COVID-19 or SARS-CoV-2 Induced Monocyte Activation by Cineole

As equally surprisingly found by the applicant, 1,8-cineole equally causes inhibition of monocyte activation induced by COVID-19 or SARS-CoV-2.

The spike protein is used as the SARS-CoV-2 pseudovirus, since it has already been described that proteins from the viral envelope elicit an immune response. This has already been described in platelets, macrophages and other cells. Consequently, it could also be shown by means of a docking study that the spike protein can bind to Toll-like receptors (TLR) and thus trigger a serious inflammatory mechanism in the cell.

In the context of the present invention, the applicant can demonstrate that 1,8-cineole inhibits SARS-CoV-2-induced monocyte activation. Since the virus protein mainly activates NFkB and MAPK signaling pathways in monocytes and these signaling pathways can be inhibited by 1,8-cineole in platelets, the present invention can particularly demonstrate that 1,8-cineole can also inhibit inflammatory signaling pathways in monocytes.

For this purpose, monocytes (untreated=comparison or pretreated with 1 mM 1,8-cineole for 1 h) are each stimulated with 5 μg/mL S1 spike protein. Total protein levels and phosphorylated protein levels of NFkB and MAPK pathways are determined by Western blot analysis.

The results show that 1,8-cineole significantly inhibits S1-activation. Mainly, the phosphorylations of IkBa, NFkB, Akt and Erk1/2 can be inhibited. Since the NFkB pathway is basically responsible for expressing pro-inflammatory genes and consequently producing and secreting pro-inflammatory cytokines, it also directly follows that cytokine secretion is also decreased under cineole.

Further experiments by the applicant on the inhibitory effect of 1,8-cineole in SARS-CoV-2-activated monocytes confirmed these results.

1-32. (canceled)

33. A method for the treatment of a COVID-19 infection,
wherein the method comprises the step of administering to a patient suffering from a COVID-19 infection a pharmaceutically effective amount of a 1,8-cineole,
wherein the 1,8-cineole is applied as a systemic dosage form which is resistant to gastric juice but soluble in the small intestine,
wherein the 1,8-cineole is present as a pure substance, wherein the 1,8-cineole is present with a purity of at least 95 wt. %, based on the 1,8-cineole, and wherein the 1,8-cineole is free of other terpenes.

34. The method according to claim 33,
wherein the comprises at least one of a prophylactic and therapeutic treatment.

35. The method according to claim 33,
wherein the 1,8-cineole is applied as a capsule, dragee, pill or tablet.

36. The method according to claim 33,
wherein the 1,8-cineole is administered at a daily dose in the range of from 10 to 2,000 mg/diem.

37. The method according to claim 33,
wherein the 1,8-cineole is administered together with at least one physiologically acceptable excipient;
wherein the physiologically acceptable excipient is at least one of miscible with and soluble in 1,8-cineole; and
wherein the physiologically acceptable excipient is present in a liquid or solid state at a temperature of 20° C. and at atmospheric pressure.

38. The method according to claim 5,
wherein the physiologically acceptable excipient is selected from the group consisting of triglycerides of C₆-C₁₂-fatty acids.

39. The method according to claim 33,
wherein the 1,8-cineole is used or applied together with at least one further active ingredient;
wherein the further active ingredient is selected from the group consisting of (i) anti-inflammatory active ingredients, (ii) blood-thinning and anticoagulant active ingredients, (iii) antiviral active ingredients, (iv) cardiovascular active ingredients, and combinations thereof.

40. The method according to claim 33,
wherein the 1,8-cineole is used for at least one of suppression and attenuation of a hyperinflammatory syndrome caused by a COVID-19 infection.

41. The method according to claim 33,
wherein the 1,8-cineole is used for at least one of suppression and attenuation of a hypercytokinemia caused by a COVID-19 infection.

42. The method according to claim 33,
wherein the 1,8-cineole is used for suppressing the replication of corona viruses occurring in thrombocytes, as caused by a COVID-19 infection.

43. The method according to claim 33,
wherein the 1,8-cineole is used for the treatment of systemic organ involvement caused by a COVID-19 infection.

44. The method according to claim 33,
wherein the 1,8-cineole is used as at least one of an inducer of NO-production and for remedying and alleviating NO-deficiency states caused by a COVID-19 infection.

45. The method according to claim 33,
wherein the 1,8-cineole is used for at least one of remedying and alleviating oxidative and inflammatory conditions in the body of a diseased patient occurring in a COVID-19 infection.

46. A drug or medicament in the form an antiviral agent for the treatment of a COVID-19 infection,
wherein the drug or medicament comprises a 1,8-cineole together with at least one physiologically acceptable excipient,
wherein the 1,8-cineole is applied as a systemic dosage form which is resistant to gastric juice but soluble in the small intestine,
wherein the drug or medicament contains the 1,8-cineole as a pure substance with a purity of at least 95 wt. %, based on the 1,8-cineole, and wherein the drug or medicament contains the 1,8-cineole free of other terpenes.

47. The drug or medicament according to claim 46,
wherein the physiologically acceptable excipient is at least one of miscible with and soluble in 1,8-cineole; and
wherein the physiologically acceptable excipient is present in a liquid or solid state at a temperature of 20° C. and at atmospheric pressure.

48. The drug or medicament according to claim 46,
wherein the physiologically acceptable excipient is selected from the group consisting of triglycerides of C₁-C₁₂-fatty acids.

49. A pharmaceutical composition for the treatment of a COVID-19 infection,
wherein the composition comprises a 1,8-cineole together with at least one physiologically acceptable excipient,
wherein the 1,8-cineole is applied as a systemic dosage form which is resistant to gastric juice but soluble in the small intestine,
wherein the composition contains the 1,8-cineole as a pure substance with a purity of at least 95 wt. %, based on the 1,8-cineole, and wherein the composition contains the 1,8-cineole free of other terpenes.

50. The pharmaceutical composition according to claim 49,
wherein the physiologically acceptable excipient is at least one of miscible with and soluble in 1,3-cineole; and
wherein the physiologically acceptable excipient is present in a liquid or solid state at a temperature of 20° C. and at atmospheric pressure.

51. The pharmaceutical composition according to claim 49,
wherein the physiologically acceptable excipient is selected from the group consisting of triglycerides of C₆-C₁₂-fatty acids.

52. The pharmaceutical composition according to claim 49,
wherein the 1,8-cineole is applied together with at least one further active ingredient, wherein the further active ingredient is selected from the group consisting of (i) corticosteroids, (ii) blood-thinning and anticoagulant active ingredients, (iii) antiviral active ingredients selected from the group consisting of Remdesivir, protein-based antiviral active ingredients, monoclonal antibodies, immunoglobulins and interferons, (iv) cardiovascular active ingredients, antihypertensives and ACE inhibitors, and combinations thereof.

53. A pharmaceutical combination for the treatment of a COVID-19 infection, wherein the pharmaceutical combination comprises at least the following components (A) and (B):
(A) a 1,8-cineole; and
(B) at least one further active ingredient selected from the group consisting of (i) anti-inflammatory active ingredients, (ii) blood-thinning and anticoagulant active ingredients, (iii) antiviral active ingredients, (iv) cardiovascular active ingredients, and combinations thereof;
wherein the 1,8-cineole is present as a systemic dosage form which is resistant to gastric juice but soluble in the small intestine,
wherein the 1,8-cineole is present as a pure substance, wherein the 1,8-cineole is present with a purity of at least 95 wt. %, based on the 1,8-cineole, and wherein the 1,8-cineole is free of other terpenes.
